(19) United States
(12) Patent Application Publication
(10) Pub. No.: US 2002/0142368 A1
(43) Pub. Date: Oct. 3, 2002

(54) Gelatin manufacturing process and product

(76) Inventors: Vesselin D. Miladinov, Forth Worth, TX (US); Aristippos Gennadios, Greensboro, NC (US); Milford A. Hanna, Lincoln, NE (US)

Correspondence Address: MACCORD MASON PLLC, 300 N. GREENE STREET, SUITE 1600, P. O. BOX 2974, GREENSBORO, NC, 27402, US

(21) Appl. No.: 09/801,524

(22) Filed: Mar. 8, 2001

(51) Int. Cl. C12P 21/06

(52) U.S. Cl. 435/68.1

(57) ABSTRACT

The present invention overcomes the deficiencies of the prior art by providing a process for manufacturing gelatin including cleaning a fowl-based source of collagen; subjecting the fowl-based source of collagen to at least one water extraction to extract gelatin from the collagen source; and separating the gelatin from any resulting by-products, wherein the process does not require an initial acid or lime pretreatment step. The present invention also includes a product made from this process.

### FIELD OF THE INVENTION

The present invention generally relates to a method for manufacturing gelatin and more specifically to a method for manufacturing fowl-derived gelatin and gelatin-based formulations that are thereby formed.

### BACKGROUND OF THE INVENTIION

Gelatin-based formulations enjoy a wide range of commercial utility. For example, gelatin-based formulations have been used in wet processed photographic emulsions, as dosage forms for medicaments, in cosmetics as a binder, and in a wide range of food products. Gelatin exhibits useful physical and chemical properties that support a broad range of utility. Gelatin is a biopolymer in high demand and its market is expected to keep growing in the near future. The current methods for manufacturing gelatin, however, present several deficiencies related to processing costs, religious conflicts and health concerns regarding gelatin sources.

Gelatin is manufactured by controlled hydrolysis of collagen, which is present in the bones, skins, and white connective tissues of animals. Commercially, the primary raw materials for gelatin manufacturing are pigskins, and bones and skins from bovine animals. Gelatin obtained from acid hydrolysis of collagen is known as Type A gelatin, whereas gelatin obtained from alkali hydrolysis of collagen is known as Type B gelatin.

Current methods for manufacturing Type A and Type B gelatin are time-extensive and require relatively expensive equipment investments. As an example of Type A gelatin manufacture, pork skins are macerated; washed to remove extraneous matter; swelled for 10 to 30 hours in 1-5% hydrochloric, phosphoric, or sulfuric acid; washed to reduce the acid; the pH is adjusted; thereafter four to five extractions are made at ever-increasing temperatures with each extraction lasting 4 to 8 hours; then the grease is removed, the gelatin solution is filtered, and, often, deionized. See Kirk-Othmer Encyclopedia of Chemical Technology, 3rd Ed., Vol. 11, pp. 713-14. For Type B gelatin, generally bones from bovine sources are crushed and degreased; the rendered bone pieces are demineralized with 4-7% hydrochloric acid for 7 to 14 days; the bones are washed and pumped to liming tanks where the bones are agitated in a lime slurry for 3 to 16 weeks; the ossein is washed for 15 to 30 hours to remove the lime and then acidified to a pH of 5-7 with hydrochloric, sulfuric, phosphoric, or acetic acid; then the extraction process, similar to that described above for Type A gelatin is performed followed by grease removal, filtration, and, often, deionization. Id. Thus, there is a need to reduce the time investment and processing costs involved in gelatin manufacture.

As noted above, current gelatin manufacture presents health and religious concerns, as well. For example, the softgel industry mainly uses gelatin derived from bovine bones due to the resulting “machinability” of the gelatin. Softgels (soft gelatin capsules) are a dosage form for the administration of liquid, semi-solid, and solid fills. The preferred gelatin-based capsule sheath composition is characterized by flexibility and a non-tacky consistency. These desired physical characteristics are based upon the formation of capsules using encapsulation machinery. One form of gelatin capsule production known in the art uses a rotary die process in which a molten mass of a gelatin-based sheath formulation is fed from a reservoir onto cooled drums to form two spaced sheets or ribbons of the gelatin-based formulation in a semi-molten state. These ribbons are fed around rollers and brought together at a convergent angle into the nip of a pair of roller dies that include opposed die cavities. The material to be encapsulated is fed into the wedge-shaped joinder of the ribbons. The gelatin ribbons are continuously conveyed between the dies, with portions of the medicament being trapped between the sheets inside the die cavities. The sheets are then pressed together, and severed around each die so that opposed edges of the sheets flow together to form a continuous gelatin-based sheath around the entrapped medicament. The part of the gelatin-based sheath that is severed from the segments forming the capsules is then collected and discarded or recycled. The soft capsules are then dried to increase the integrity of the sheath, and packaged for later distribution and consumption. Manufacture of uniform soft gelatin capsules requires a sheath material that has good “machineability,” i.e., it is important that the sheath material be of a non-tacky or non-sticky nature, so that the sheath material can be brought into contact with the rollers without sticking. Further, if the gelatin-based formulation is highly viscous, “thick,” and/or tough, it will also affect the machineability of the gelatin sheath material on the encapsulating machine. Other encapsulating machines equally applicable for gelatin-based formulations, such as hard tablet enrobing, disclosed in U.S. Pat. Nos. 5,146,730 and 5,549,983, hard gelatin shells, and gelatin-dipped products require that the gelatin have certain physical characteristics for proper machineability as well. Thus, when developing a more cost-effective method for manufacturing gelatin, the resulting gelatin formed should exhibit substantially similar physical properties for commercial applicability.

While bovine and porcine starting materials provide adequately machineable gelatin, large groups around the world cannot ingest the resulting gelatin products. Certain religious and dietary concerns prohibit the ingestion of procine and bovine products by certain groups, e.g., vegetarians, Hebrews, Muslims, Hindus, etc.

More recently, the sources of bovine and porcine starting materials has raised some concern. For example, bovine spongiform encephalopathy (BSE), or “mad cow disease” (particularly in the United Kingdom) has adversely affected the gelatin manufacturing industry. Further, the sun-dried bones of cattle dying from natural causes in India and Pakistan have lost some level of commercial acceptance due to lack of traceability. As a result, bone supplies for gelatin production have shrunk and prices have increased. Additionally, the potential for continued downturns in the consumption of beef and pork, or the reduction in beef and pork production because of reduced profitability, would portend for additional upward pressure on the price of gelatin derived from these products.

There is a need, therefore, for a method of manufacturing gelatin, preferably from a non-bovine or non-porcine source, that will reduce the overall processing costs thereby increasing the economic feasibility of gelatin processing.

### SUMMARY OF THE INVENTION

The present invention overcomes the deficiencies of the prior art by providing a process for manufacturing gelatin including cleaning a fowl-based source of collagen; subjecting the fowl-based source of collagen to at least one water extraction to extract gelatin from the collagen source; and separating the gelatin from any resulting by-products, wherein the process does not require an initial acid or lime pretreatment step. The present invention also includes a product made from this process. In this manner, the present invention will reduce the overall processing costs of gelatin to increase the economic feasibility of gelatin processing.

These and other aspects of the present invention as disclosed herein will become apparent to those skilled in the art after a reading of the following description of the preferred embodiments.

### DETAILED DESCRIPTION OF THE PREFERRED EMBODIMENT

The present invention provides an improved process for manufacturing gelatin that overcomes the above-noted deficiencies in the current gelatin manufacturing art.

As used herein, the term “gelatin” should be interpreted to include gelatin-based formulations as well. In other words the present invention should not be interpreted to limit the end-use of the gelatin. Rather, the present invention provides gelatin that is viable for a variety of commercial applications.

The present invention preferably begins with cleaning the source of collagenous protein. In order to avoid the above-noted potential deficiencies regarding health, religious, and cost concerns regarding porcine and bovine collagen sources, the present invention uses a fowl-based source of collagen. As used herein, the term “fowl” includes any bird whose bones, skin, flesh, feet, deboned residue, organ membranes, or connective tissue is approved for use as a source for edible material. Particularly, the term includes poultry, i.e., domestic fowls such as chickens, turkeys, ducks, and/or geese.

Fowl, preferably poultry, is an improved source of collagen due to the increasing demand for value-added fowl co-products, namely, products manufactured from fowl skins, bones, deboned residues, and organ membranes. Additionally, fowl-based collagen overcomes the above-mentioned deficiencies regarding current health concerns regarding tainted beef products as well as overcoming certain religious concerns.

The process of the present invention includes subjecting the fowl-based source of collagen to at least one water extraction to extract gelatin from the collagen source. Importantly, the process of the present invention does not require an initial acid or lime pretreatment step. The gelatin is obtained from the hydrolysis of the fowl-based collagen through direct extraction. Without being limited to any particular theory, the present inventors believe the direct extraction of gelatin from fowl bones and skins is possible because fowl-based collagen is less cross-linked than mammalian collagen. Therefore the fowl-based collagen is more susceptible to hydrolysis breakdown.

The elimination of the acid or lime pretreatment steps increases the economic feasibility of this gelatin manufacturing process. There are several notable benefits of the present invention over the prior manufacturing processes described above for Type A and Type B gelatins. For example, the overall processing cycle is shortened. The requirements for separate pretreatment equipment and facilities (e.g., liming pits) are eliminated. Further, the added expense of lime and/or acid starting materials and storing facilities therefore is eliminated. Additionally, the water effluent treatment requirements are reduced.

The present invention includes one or more hot water extractions. Surprisingly, the present inventors have found that the water extractions may be performed at temperatures below 75° C. Thus, contrary to prior collagen hydrolysis processes, the present invention does not require the extreme high temperatures, i.e. about 212° C. or above, in order to manufacture a commercially viable gelatin product. As noted above, the present inventors believe that the reduced cross-linking of fowl-based collagen allows for direct extraction. The temperatures at which the present invention is successful, however, is surprising in light of the prior gelatin manufacturing methods that involved more extreme temperatures.

Preferably, each of the extractions is carried out for about 30 to about 300 minutes. Most preferably, the total extraction time is under about (300 × preferred # of extractions) minutes. Thus, the present invention provides a more time-effective method for producing gelatin than heretofore known.

Preferably, three extractions are performed. In more detail, the first extraction is for approximately 3 hours in 55 to 70° C. water at atmospheric pressure. The second extraction is for approximately 3 hours in 70 to 85° C. water under atmospheric pressure. The third extraction is for approximately 4 hours in 85 to 96° C. water under atmospheric pressure. Alternatively, a single extraction at a temperature of 120° C. under sub-atmospheric pressure may be performed.

After one or more extractions, the gelatin is separated from any resulting by-products, such as grease and non-collagenous proteins. A preferred method for separating the gelatin from the by-products includes decanting the grease and centrifuging to provide product streams of gelatin, grease, and non-collagenous proteins. For example, from centrifugation of skin extractions, two streams, a stream of gelatin and a stream of grease and non-collagenous proteins, are obtained. With centrifugation of bone extractions, three product streams, namely, gelatin, grease, and non-collagenous proteins, are obtained. Optionally, the fowl-based collagen source may be pre-treated with proteolytic enzymes prior to extraction to remove any non-collagenous proteins.

The source of fowl-based collagen should be cleansed prior to performing the extraction steps noted above. Preferably, the collagen source is pasteurized by steam pasteurization as is known in the art.

Gelatin yields using the present invention are about 3.0 to 3.5% by weight, on a wet basis.

The present invention also includes the product formed by the process herein described. Thus, the present invention includes a composition comprising gelatin hydrolyzed from a collagen source without the need for acid or lime treatment through at least one water extraction, wherein each of the at least one extractions is performed at a temperature under about 75° C. Again, the source of the collagen is fowl. The resulting gelatin composition derived from the described mild heat treatment has a Bloom strength in the range of about 70 to about 220. Thus, the resulting gelatin is particularly useful for capsule manufacturing due to the high jelly strength. Additionally, due to the elimination of the acid and/or the lime pretreatment step, the unwanted breakdown of fat into fatty acids, which emulsify with the gelatin, is avoided. In short, the resulting gelatin product may provide commercial advantages not heretofore available using current manufacturing processes.

### EXAMPLE 1

Chicken skins were subjected to the described process. Three successive extractions in water were performed at respective temperatures of about 60, 75, and 96° C. Each extraction lasted approximately 3 hours. The gelatin obtained from each extraction was evaluated for Bloom strength, viscosity, melt point, and set point. Each of these parameters were determined using aqueous gelatin solutions with a gelatin concentration of 6.67% by weight. The following results were obtained:

| Property | Extraction #1 | Extraction #2 | Extraction #3 |
|---|---|---|---|
| Bloom strength | 176 | 123 | 72 |
| (grams) | | | |
| Viscosity (milliPoise) | 34.8 | 27.8 | 31.8 |
| Meltpoint (° C.) | 38 | 37 | 34 |
| Setpoint (° C.) | 23 | 22 | 21 |

Bovine/porcine gelatins used currently for softgel manufacturing typically range in Bloom strength from 150 to 250 grams and in viscosity from 25 to 45 milliPoise (See, Stanley, J. P., The Theory and Practice of Industrial Pharmacy, ₃rd edition. L. Lachman, H. A. Lieberman, and J. L. Kanig, editors, Philadelphia, Pa.: Lea & Febiger, pp. 398-412, 1986).

### EXAMPLE 2

Turkey skins were processed according to the process described above in Example 1 and extracted in water at 60° C. for 3 hours. The gelatin obtained had a Bloom strength of 123 grams and a viscosity of 39 milliPoise (both parameters again determined using aqueous gelatin solutions with a gelatin concentration of 6.67% by weight).

Thus, the gelatin resulting from the present invention appears to present a viable alternative to beef and pork based gelatins.

Although specific embodiments of the present invention have been illustrated and described in detail, it is to be expressly understood that the invention is not limited thereto. The above detailed description of the embodiment is provided for example only and should not be construed as constituting any limitation of the invention. Thus, modifications will be obvious to those skilled in the art, and all modifications that do not depart from the spirit of the invention are intended to be included within the scope of the appended claims.

What is claimed is:

1. A process for manufacturing gelatin comprising:
cleaning a fowl-based source of collagen;
subjecting the fowl-based source of collagen to at least one water extraction to extract gelatin from the collagen source; and
separating the gelatin from any resulting by-products, wherein the process does not require an initial acid or lime pretreatment step.

2. The process of claim 1 wherein each of the at least one water extractions is performed at temperatures below about 96 ° C.

3. The process of claim 1 further comprising:
pasteurizing the fowl-based source of collagen.

4. The process of claim 3 wherein the pasteurizing is accomplished through treatment with steam.

5. The process of claim 1 wherein the fowl-based source of collagen is subjected to 3 extractions.

6. The process of claim 1 wherein 3 extractions are performed and wherein the first extraction is for approximately 3 hours in about 55-70° C. water at atmospheric pressure, the second extraction is for approximately 3 hours in about 70-85° C. water at atmospheric pressure, and the third extraction is for approximately 4 hours in 85-96° C. water under atmospheric pressure.

7. The process of claim 1 wherein the fowl-based source of collagen is subjected to one extraction at a temperature of approximately 120° C under. sub- atmospheric pressure.

8. The process of claim 1 wherein each of the at least one extractions lasts for about 30 to about 300 minutes.

9. The process of claim 1 wherein the total extraction time is under about (300 ×preferred number of extractions) minutes.

10. The process of claim 1 wherein the by-products are grease and non-collagenous proteins.

11. The process of claim 1 wherein separating further comprises:
decanting grease; and
centrifuging.

12. The process of claim 1 further comprising:
treating the fowl-based collagen source with proteolytic enzymes prior to extraction to remove non-collagenous proteins.

13. The process of claim 1 wherein the fowl-based collagen source is poultry skins, bones, and feet tendons.

14. The product formed by the process of claim 1.

15. A composition comprising:
gelatin hydrolyzed from a collagen source without the need for acid or lime treatment through at least one water extraction, wherein each of the at least one extractions is performed at a temperature under about 96° C.

16. The composition of claim 15 wherein the source of collagen is fowl.

17. The composition of claim 15 wherein the resulting gelatin has a Bloom strength of about 70 to about 220.

18. The composition of claim 15 wherein the at least one water extraction further comprises:
a first extraction is for approximately 3 hours in about 55-70° C. water at atmospheric pressure;
a second extraction is for approximately 3 hours in about 70-85° C. water at atmospheric pressure; and
a third extraction is for approximately 4 hours in 85-96° C. water under atmospheric pressure.

19. A softgel comprising a gelatin shell wherein said gelatin is gelatin hydrolyzed from a collagen source without the need for acid or lime treatment through at least one water extraction, wherein each of the at least one extractions is performed at a temperature under about 96° C.
